(19) United States
(12) Patent Application Publication
(10) Pub. No.: US 2019/0015565 A1
(43) Pub. Date: Jan. 17, 2019

(54) METHOD AND SYSTEM FOR ELIMINATING SURGICAL SUCTION NOISE IN AN OPERATING ROOM

(71) Applicants: Linda Louise MYERS, Lancaster, PA (US); Earl Frederick MYERS, III, Lancaster, PA (US)

(72) Inventors: Linda Louise MYERS, Lancaster, PA (US); Earl Frederick MYERS, III, Lancaster, PA (US)

(21) Appl. No.: 15/648,723

(22) Filed: Jul. 13, 2017

(51) Int. Cl. A61M 1/00; A61M 39/28

(52) U.S. Cl. CPC A61M 1/0045; A61M 2205/42; A61M 39/284

(57) ABSTRACT

A method and system of and for aspirators and/or medical suction methods and systems. Surgical suction methods and systems including a clamp for restricting or occluding any flexible medical suction tubing being used.

### FIELD OF THE INVENTION

The present invention relates to aspirators and/or medical suction methods and systems. More specifically, the present invention relates to surgical suction methods and systems comprising eliminating the suction sound in an operating unit using a clamp for occluding any flexible medical suction tubing being used.

### BACKGROUND OF THE INVENTION

In the majority of all surgical procedures performed worldwide, on a daily basis, surgical suction systems are used to evacuate surgical plume from surgical cauterizing systems, to evacuate blood, and to evacuate irrigation fluids away from a surgical field. Such known surgical suction systems typically are individual, stand-alone, electrically powered vacuum systems positioned close to, but outside a sterile surgical field. Sterile surgical suction tubing is attached to the suction system thus allowing for use, and making available within a sterile surgical field so-called “on demand” suction. As depicted in PRIOR ART FIG. 1, typically known dental, airway, and/or surgical suction systems 10 comprise a detachable collection reservoir 12, an air inlet/outlet reservoir lid 14, a proximal air outlet 16, a distal air/fluid/specimen inlet 18, and a flexible suction tubing 104.

In operation, in such known suction systems 10, a vacuum source (not shown) is connected to the proximal air outlet 16 of the reservoir lid 14 creating a desired partial vacuum in the detachable collection reservoir 12, in the distal air/fluid/specimen inlet 18; and subsequently, producing a desired partial vacuum in any detachable suction tubing 104. In the case of surgical suction, a flexible suction tubing 104 is intended and used to cross into a sterile surgical field 20 and to an operative site 22 about and within a patient 24. As desired by a practitioner, a flexible suction tubing 104 may be used directly at an operative site 22, or a suction attachment 26 may be connected and used.

Ideally, adequate airway, field, and surgical wound suction is reliably and immediately available for use by medical practitioners and staff during a procedure. The suction is available and turned on throughout the medical procedure so that it is available “on-demand.” However, most known surgical suction systems produce a substantial amount of noise while enabled, more specifically when not directly being used to evacuate an operative site or the like during a procedure.

In the modern day operating room, the suction system is required to be turned on and available “on-demand” throughout the medical procedure. Furthermore, the background noise caused by the suction system is undesirable and can be very distracting to medical staff. In particular, lower-volume long-duration background noise, such as that produced while a suction system is enabled but not being used at an operative site, and higher-volume shorter-duration noise, such as that produced while an operative field is being suctioned or evacuated. Both types of “suction noise” can interfere with communication—posing a safety hazard to patients.

Suction noise is primarily caused by system vacuum pumps, which are typically driven by an electric motor, or by piped in vacuum systems and/or caused by the diameter and configuration of a hose or tubing, and any attachments being used at the operative site.

When a surgical suction system is powered on for use, and suction tubing is attached from a sterile field, a constant, loud, hissing sound is created. This hissing sound greatly impedes verbal communication within a surgical team, and is an overall annoyance tantamount to noise pollution. There are known professional and academic articles addressing loud noise in an operating room leading to increased surgical site infection (see http://www.mdedge.com/acssurgerynews/article/98123/general-surgery/noisy-or-linked-communication-gaps-surgical-site, set forth as Exhibit D to the Appendix).

A few factors come into play eliminating or reducing surgical suction noise caused by suction tubing and attachments. As a general function of fluid dynamics, the inner diameter of a conduit and the rate or volume of gas either entering or exiting any conduit orifice will determine the pitch and volume of any audible frequencies produced. Similar to a musical instrument, the sound produced depends on how hard a musician blows into it, and the overall configuration of the instrument itself.

Typically, when drawing air into a conduit, such as medical suction tubing or attachments; the greater volume and speed of air being “sucked in”, the greater the noise produced. To limit noise while providing adequate airway or surgical suction, tubing and/or hoses are designed having a relatively large diameter, and any vacuum draw maintained at a flow rate as low as possible. Generally, airway and surgical suction tubing is 3/16″ to ½″ in diameter and such systems range from 5″-to-25″ Hg of vacuum produced.

Existing systems include: U.S. Pat. Nos. 4,036,232, 4,560,378, 5,035,399, 5,318,546, 5,419,769, 6,196,519, 6,644,618, 7,434,779, 8,523,828, AND U.S. 20080051731.

Other existing systems include suction canister(s), typically configured as a single unit, or on a quad “carousel.” These systems sit outside the sterile field, sterile surgical suction tubing connects to it from sterile field. Such systems are powered by vacuum system from OR wall unit and cannot be touched by surgical team during procedure (i.e. they are not sterile.). An example of the system is shown as Exhibit A in the Appendix to the Specification.

Another existing system includes a stand-alone, electrically powered, self-contained Zimmer Dornoch(TM) unit. This system sits outside the surgical field and a sterile surgical suction tubing connects to it from sterile field. This system cannot be touched by surgical team during procedure. (i.e. is not sterile). An example of the system is shown as Exhibit B in the Appendix to the Specification.

Relatedly, known aspirator and/or suction tips or attachments include: vallfors or “split tips”, Yankauer tips (large opening with vented bulbous head), maxi tips (split lumen), narrow opening (“whistle tips”), and others. As with any suction tubing itself, any suction attachment inlet diameter will impact the noise produced when the system is enabled and when used.

In an attempt to eliminate suction noise caused by “pump-in-room” systems, many times the system is simply deactivated when not immediately being used at an operative site. However, this is not best practice, typically highly disadvantageous as a practitioner at each use must have the circulating nurse a scrub-person activate and deactivate the suction system via controls outside the sterile field. As with many surgical systems, a foot-switch may be included with a suction system for unaided control by a practitioner; however this is also highly undesirable as such adds to the plurality of controls under a practitioner's feet.

In another attempt to eliminate or reduce suction noise by “on demand” system actuation, pistol grip or fire nozzle type attachments including a valve and system on/off control are available. However continual clogging is at issue with these types of hand pieces. Moreover, such attachment/control systems are prohibitively expensive, and are rarely used.

Based upon first hand instant inventor knowledge and experience, in reality the state of the art involves a surgical scrub person attempting to eliminate suction tip noise or hiss:

a) by bending a suction tube in one hand as one would a “pinch-off” a garden hose; however this reduces the scrub person to only one hand for other uses—which is impractical and potentially unsafe.

b) more commonly by, placing and crimping a precision surgical clamp across a suction tube which may and does damage the expensive surgical instrument rendering it unfit for its intended purpose and unsafe for use on or with future patients.

This known and common “surgical instrument crimping via surgical instrument intended for another medical purpose” technique is additionally highly disadvantageous in that such creates a circumstance which hinders the overall progress of a surgical procedure. When a scrub person applies a surgical clamp to a suction tube, the clamp is usually placed proximal or closest to the scrub person, who is located at the foot and side of a surgical operating room table, and away from the surgeon (˜1-2 ft.). When a surgeon requires surgical suction for a patient, they must “demand it” and rely on a scrub person to unclamp the suction tubing. This greatly hinders a scrub person who is or may be otherwise occupied in their scrub role, and hinders a surgeon or surgeon assistant who may wish to but cannot accomplish required suction the instant it is needed. Such surgical suction is neither “on demand” nor “at hand” for a surgeon.

Primarily, the occasions when surgical suction is required tends to be in bleeding situations, where time is critical and delays costly. While a surgeon waits for “on demand” suction to become available using known systems and techniques, manual pressure must be applied to a “bleeder”, including arterial bleeders, which is dangerous to a patient in the extreme. Repeated if not constant waiting for surgical suction caused by known systems inadequacy also results in prolonging operative procedures and in unwanted and prolonged patient time under general anesthesia.

It is desired to provide a method and system that solves the disadvantages in the prior art, which do not adequately solve the problem of eliminating the hissing suction noise in the operating room.

### SUMMARY OF THE INVENTION

It is an object of the present invention to provide a method and system for eliminating noise inherently produced by a vacuum placed on a medical suction orifice, the method and system including a clamp for restricting and/or occluding a portion of flexible medical suction tubing.

It is an object of the present invention to provide a clamp for and by placing control of surgical suction in surgeon's hand for immediate use, thereby eliminating the need for the scrub person to control the suction by any method. Certain objects of the present invention use a surgical clamp or other medical instrument to perform this method.

It is an object of the present invention to eliminate the need of the circulating nurse to monitor the non-sterile suction unit, as the circulating nurse is often much too busy with the legal documentation, and often times must leave the room altogether, and so is unavailable to operate the suction unit outside the sterile field. This is itself a menial, unnecessary task, which impedes the efficiency of the procedure, and overall flow of the operating room.

It is an object of the present invention to provide a method and system for eliminating noise inherently produced by a vacuum placed on a medical suction attachment orifice, the method and system including a clamp for restricting and/or occluding a flexible medical tubing portion integral to and/or with the attachment.

It is an object of the present invention to provide a method and system of and for placing control of the suction in the surgeon's hand for immediate use, thereby eliminating the need for the scrub person to clamp it off with any surgical instrument.

It is an object of the present invention to provide a clamp for eliminating noise inherently produced by a vacuum placed on a medical suction orifice, wherein the clamp is sterilizable.

It is an object of the present invention to provide a clamp for eliminating noise inherently produced by a vacuum placed on a medical suction orifice, wherein the clamp is disposable.

It is an object of the present invention to provide a clamp for eliminating noise inherently produced by a vacuum placed on a medical suction orifice, wherein the clamp is made of a polymer or plastic.

It is an object of the present invention to provide a clamp for eliminating noise inherently produced by a vacuum placed on a surgical suction orifice, wherein the clamp is made of a metal or metal alloy.

It is an object of the present invention to provide a clamp for eliminating noise inherently produced by a vacuum placed on a surgical suction orifice, wherein the clamp is sterilizable.

It is an object of the present invention to provide a clamp for eliminating noise inherently produced by a vacuum placed on a medical suction orifice, wherein the clamp is captive to a surgical suction tubing or attachment.

It is an object of the present invention to provide a clamp for eliminating noise inherently produced by a vacuum placed on a medical suction orifice, wherein the clamp is not detachable from a surgical suction tubing or attachment.

It is an object of the present invention to provide a clamp for eliminating noise inherently produced by a vacuum placed on a surgical suction orifice, wherein the clamp may be enabled by and used with one hand.

These and other objects of the invention are achieved by providing a method of and for eliminating noise inherently produced by a vacuum source during a medical procedure, the method comprising the steps of: providing a clamp; connecting the surgical clamp to sterile surgical tubing; and applying force to the clamp, such that the application of force causes the clamp to transition from an open position to a first closed position, thereby eliminating noise caused by the vacuum created through the sterile surgical tubing during the medical procedure.

In certain embodiments, the method further comprises sterilizing the clamp prior to the initiation of the surgical procedure.

In certain embodiments, the method further comprises maintaining the clamp in a sterile field through the entire surgical procedure.

In certain embodiments, the first closed position of the clamp causes the diameter of the sterile surgical tubing to be reduced, but not entirely closed.

In certain embodiments, the method further comprises repositioning the clamp along the length of the sterile surgical tubing.

In certain embodiments, repositioning the clamp along the length of the sterile surgical tubing involves sliding the clamp along the length of the sterile surgical tubing.

In certain embodiments, the surgical clamp is configured to close, and wherein upon closing of the clamp, the sterile surgical tubing maintains a vacuum within the tubing while eliminating any noise caused by the vacuum.

In certain embodiments, the clamp eliminates noise from surgical procedure.

In certain embodiments, the clamp is configured to be opened and closed via a thumb of an operator.

In certain embodiments, the clamp comprises: a distal end, a hollow body, a proximal end, a bottom surface between the distal and the proximal end, a vertical arm extending from the distal end, the vertical arm having an inner face with a plurality of teeth, a horizontal arm extending from the proximal end toward the distal end, the horizontal arm having a beveled edge proximal to the inner face, a first protrusion on the bottom surface extending within the hollow body, and a second protrusion on the horizontal arm extending within the hollow body; whereby providing a first force applied to the hollow body such that the horizontal arm and the vertical arm converge to engage the beveled edge with a backside of one of the plurality of teeth, to transition the clamp from an open position to a first closed position of a plurality of plurality positions for restricting or occluding a portion of a medical suction tubing.

In certain embodiments, the clamp is serializable.

In certain embodiments, the clamp is disposable.

In certain embodiments, the clamp is made of materials selected from a group consisting of a polymer or plastic, a metal or metal alloy or a combination thereof.

In certain embodiments, the clamp is configured to be captive to the medical suction tubing.

In certain embodiments, the clamp is operable using one hand.

Other embodiments of the invention are achieved by a system for eliminating noise inherently produced by a vacuum source during a medical procedure, the system comprising a clamp, wherein the clamp includes; a distal end, a hollow body, a proximal end, a bottom surface between the distal and the proximal end, a vertical arm extending from the distal end, the vertical arm having an inner face with a plurality of teeth, a horizontal arm extending from the proximal end toward the distal end, the horizontal arm having a beveled edge proximal to the inner face, a first protrusion on the bottom surface extending within the hollow body, and a second protrusion on the horizontal arm extending within the hollow body; whereby a first force applied to the hollow body such that the horizontal arm and the vertical arm converge to engage the beveled edge with a backside of one of the plurality of teeth, to transition the clamp from an open position to a first closed position of a plurality of closed positions for restricting or occluding a portion of a medical suction tubing.

In certain embodiments, the surgical clamp is serializable.

In one exemplary embodiment, a method and system of and for eliminating noise inherently produced by a vacuum placed on a medical suction orifice, comprising a suction tube clamp including a hollow body with a distal end, a proximal end, and a bottom surface between the distal and proximal ends, wherein a vertical arm extends from the distal end having an inner face with a plurality of teeth. A horizontal arm may extend from the proximal end toward the distal end having a beveled edge proximate to the inner face of the vertical arm. A first protrusion on the bottom surface may extend within the hollow body and a second protrusion on the horizontal arm may extend within the hollow body.

In another inventive embodiment, a system of and for eliminating noise inherently produced by a vacuum placed on a medical suction orifice, includes a clamp with a hollow body having a distal end, a proximal end, and a bottom surface between the distal and proximal ends. The distal end may have a first aperture, and the proximal end may have a second aperture. A vertical arm may extend from the distal end and have an inner face with a plurality of teeth. A horizontal arm may extend from the proximal end toward the distal end. The horizontal arm may have a beveled edge proximate the inner face of the vertical arm. A first protrusion on the bottom surface may extend within the hollow body and a second protrusion on the horizontal arm may extend within the hollow body. Medical tubing may be received through the first aperture and the second aperture.

Other objects of the invention and its particular features and advantages will become more apparent from consideration of the following drawings and accompanying detailed description. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

PRIOR ART FIG. 1 is a schematic diagram of typically know medical suction systems.

FIG. 2 is a diagram of an embodiment of the inventive system and method comprising a flexible medical tubing and clamp.

FIG. 3A is a diagram of an embodiment of the inventive system and method comprising a flexible medical tubing and clamp.

FIG. 3B is a diagram of an embodiment of the inventive system and method comprising a flexible medical tubing and clamp.

FIG. 3C is a diagram of an embodiment of the inventive system and method comprising a flexible medical tubing and clamp.

FIGS. 4A-4B are photographs of an embodiment of the inventive system and method comprising a flexible medical tubing and clamp.

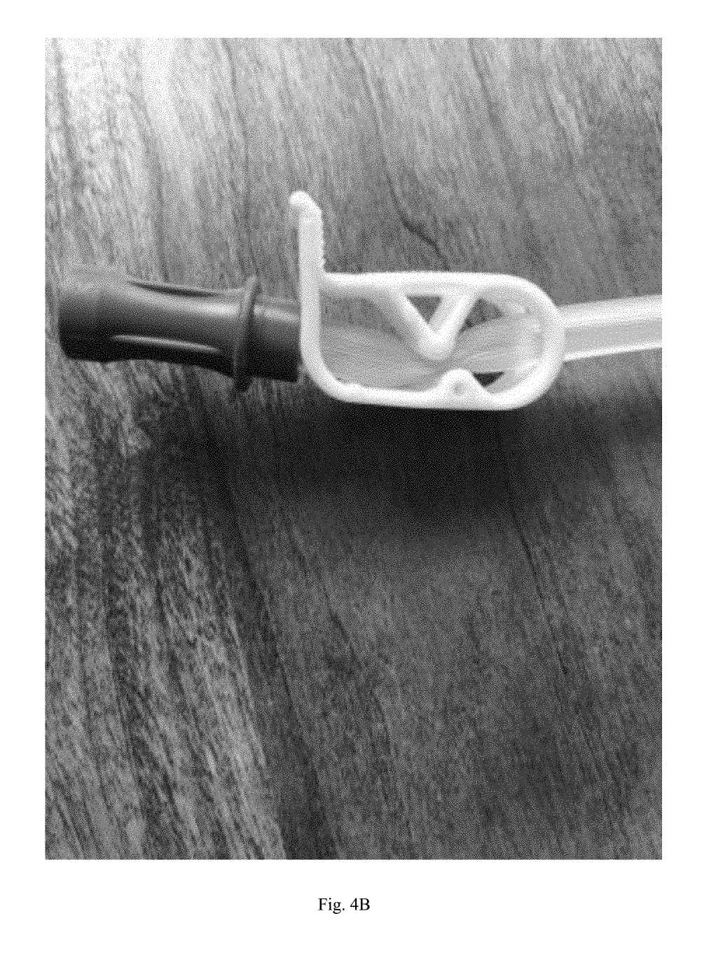

### DETAILED DESCRIPTION OF THE INVENTION

This application incorporates by reference U.S. patent application Ser. No. 13/941,039, filed Jul. 7, 2013, entitled SYSTEM, METHOD, AND APPRARTUS FOR SILENT SUCTION TUBING, published as U.S. Patent Application Publication No. 2015/0018783 A1. The contents of this application are herein incorporated by reference in its entirety.

This application incorporates the contents of the Appendix to the Specification into this application.

In the following description, numerous details are set forth for the purpose of explanation. However, one of ordinary skill in the art will realize that the invention may be practiced without the use of these specific details.

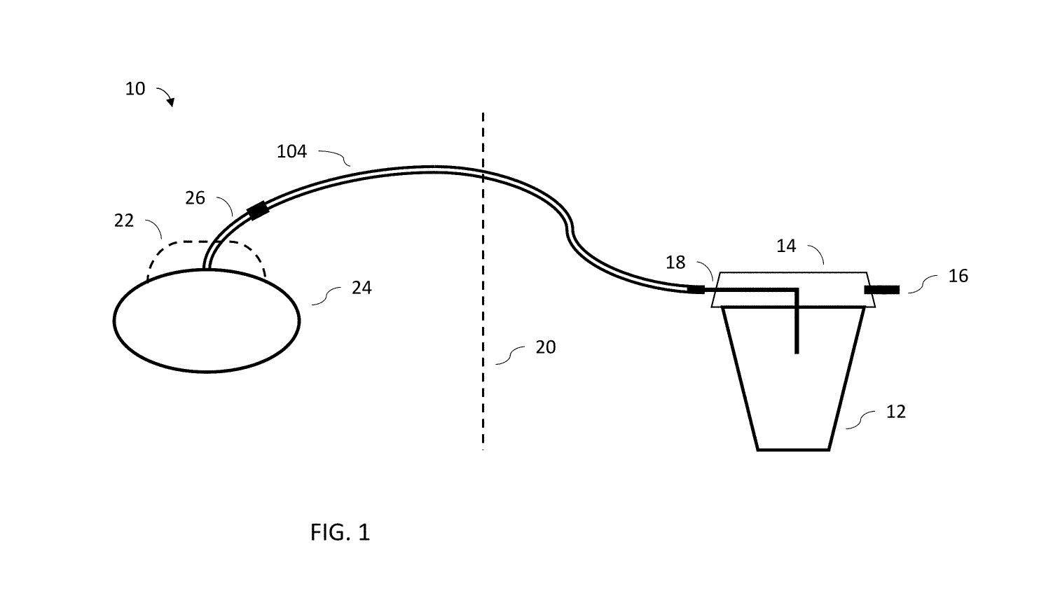

As depicted in FIG. 2, one embodiment of the inventive method and system 100 includes a clamp 102 and flexible surgical tubing 104. The surgical tubing 104 may comprise medical grade plastic or similar material as is widely understood by a person of ordinary skill in the art. The surgical tubing 104 may be coupled to a vacuum source (not shown) at a proximal end and at a distal end to a suction attachment 26, and operable to draw fluid through the suction attachment 26 and away from an operative field 22 during an operation.

As depicted in FIG. 2, the clamp 102 may have a distal end 106, a hollow portion 107 and a proximal end 108. The surgical tubing 104 may be received through distal end 106, hollow portion 107, and proximal end 108 such that distal end 106 is upstream from a suction direction of proximal end 108.

In one exemplary embodiment, distal end 106 and proximal end 108 may have complimentary apertures to allow surgical tubing to be received through the clamp 102. The apertures may be square, rectangular, circular, or any shape allows passage of the surgical tubing 104 as would be easily understood by a person of ordinary skill in the art.

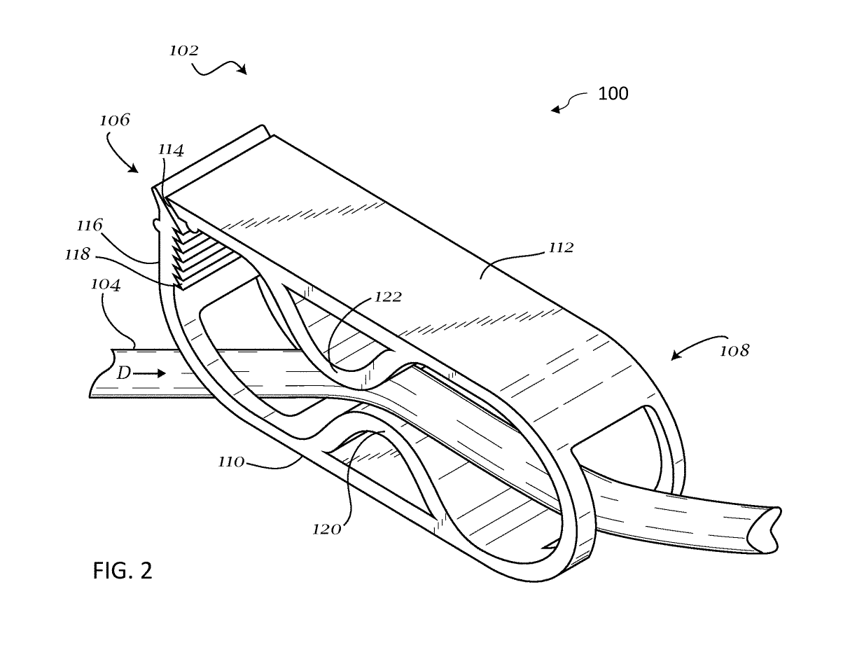

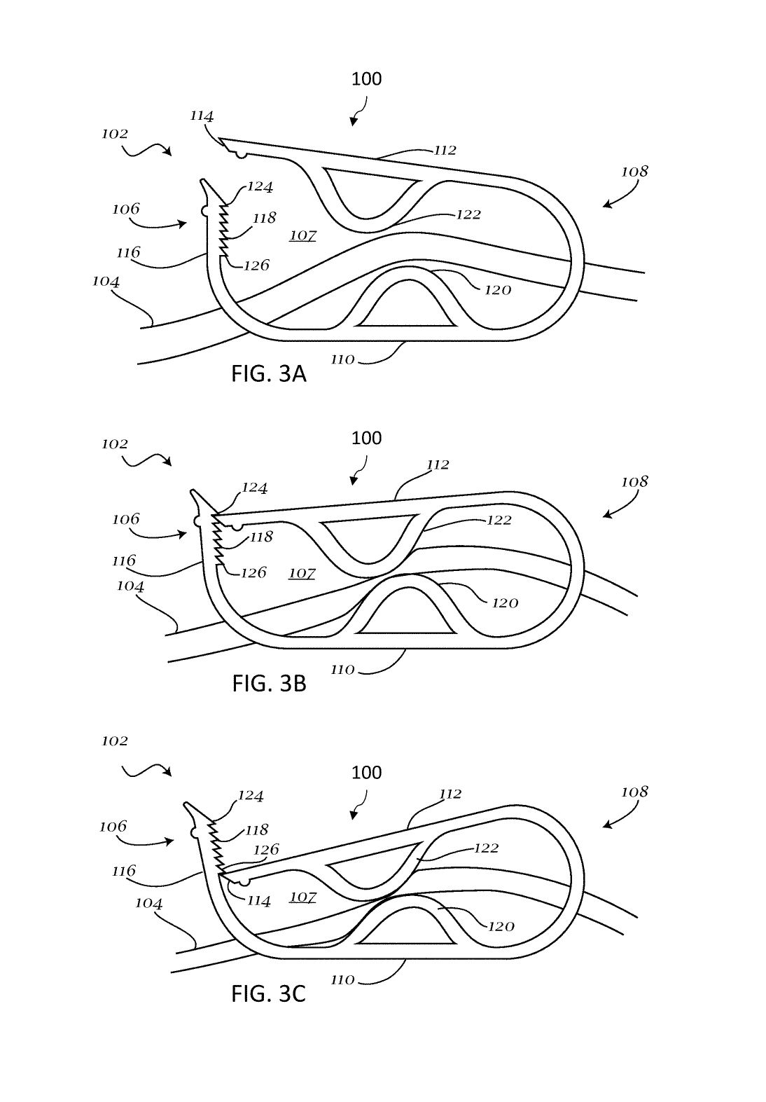

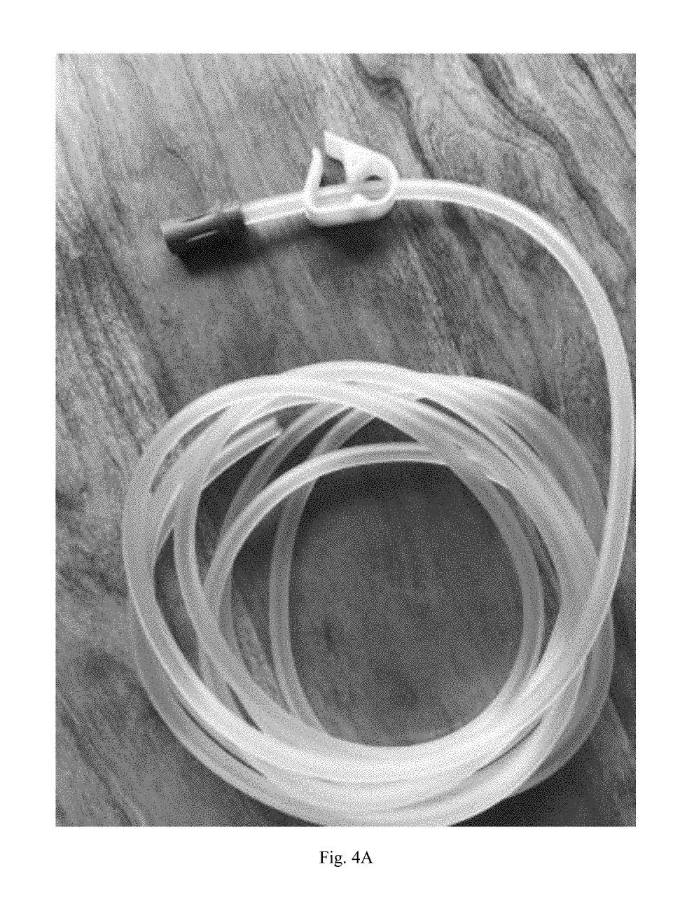

As depicted in FIG. 3A, in some exemplary embodiments, the clamp 102 may be operable to slide along a length of the surgical tubing 104.

In other exemplary embodiments, connecting end pieces may be added to the surgical tubing 104 on either side of clamp 102, to prevent the clamp 102 detachment from the surgical tubing 104 and possibly misplaced in the operative field 22 or wound. The clamp 102 may have a bottom surface 110 between respective bottom portions of the distal end 106 and the proximal end 108.

In some exemplary embodiments, the distal end 106 and proximal end 108 may have substantially rounded edges, and the bottom surface 110 may be substantially elongated and flat such that the clamp 102 may have a generally oval or oblong shape between distal end 106 and proximal end 108. A horizontal arm 112 may extend from a top portion of the proximal end 108 toward distal end 106. In some exemplary embodiments the horizontal arm 112 may be flexible but resilient with respect to proximal end 108. The horizontal arm 112 may have a distal end with beveled edge 114. A vertical arm 116 may extend from a top portion of the distal end 106. The vertical arm 116 may be substantially perpendicular to the horizontal arm 112. The vertical arm 116 may have a plurality of teeth 118. The plurality of teeth 118 may be, for example, triangular teeth which slope in a downward direction. In alternative embodiments, the plurality of teeth 118 may be any contemplated shape or size without deviating from the scope of the present invention. The plurality of teeth 118 may be on the distal end 106 and on an inner face of the vertical arm 116. The plurality of teeth 118 may be proximal to the beveled edge 114 of the horizontal arm 112. The bottom surface 110 may have a first protrusion 120 between the distal end 106 and the proximal end 108 that may extend substantially toward and within a hollow portion 107. The horizontal arm 112 may have a second protrusion 122 between the beveled edge 114 and proximal end 108 that may extend substantially toward and within the hollow portion 107.

In one exemplary embodiment, a first protrusion 120 and a second protrusion 122 may be substantially rounded. In alternative embodiments, the first protrusion 120 and the second protrusion 122 may be of any configuration, size, or shape without deviating from the scope of the present invention. The first protrusion 120 may be substantially opposite the second protrusion 122. For example, the first protrusion 120 and the second protrusion 122 may be situated on opposite sides of the surgical tubing 104. In one exemplary embodiment, the first protrusion 120 and the second protrusion 122 may be in contact with the surgical tubing 104. As explained in more detail below, the horizontal arm 112 and the vertical arm 116 may be manipulated such that a distance between the first protrusion 120 and the second protrusion 122 is modified. For example, the distance between the first protrusion 120 and the second protrusion 122 may be modified to increase or decrease a force, for example, a pinching or compressing force, exerted on the surgical tubing 104.

Referring now to FIGS. 3A-C, an inventive clamp 102 is depicted in a variety of exemplary positions. For example, FIG. 3A depicts the clamp 100 in an open position, that is the beveled edge 114 of the horizontal arm 112 is not in contact with any of the plurality of the teeth 118 of the vertical arm 116. In this exemplary embodiment, the first protrusion 120 and the second protrusion 122 may be a maximum distance apart, and may exert a minimal, if any, force on the surgical tubing 104. Thus, in the open position of FIG. 3A, suction through the surgical tubing 104 may be substantially unimpeded. Further, the clamp 102 may be free to slide along a length of the surgical tubing 104.

FIG. 3B depicts the clamp 102 in a first closed position. For example, in the first closed position, the beveled edge 114 of the horizontal arm 112 may engage a first tooth 124 of said the plurality of teeth 118 of the vertical arm 116. In this exemplary embodiment, the beveled edge 114 may act, for example as a pawl, and the plurality of teeth 118 may act, for example, as a rack, as would be understood by a person of ordinary skill in the art. As such, the interaction of the beveled edge 114 and the plurality of teeth 118 may be comparable to an open cable tie, zip tie, or similar ratcheting type fastener.

For example, to transition from an open position as depicted in FIG. 3A to the first closed position depicted in FIG. 3B, the beveled edge 114 may contact the first tooth 124. A downward force may be exerted on the horizontal arm 112, such that the beveled edge 114 may slide down the downward slope of the first tooth 124 and engage a backside of the first tooth 124. For example, a surgeon or other medical professional may exert a downward force by pressing the horizontal arm down with their finger or thumb. In this position, the backside of the first tooth 124 may act against a resilient force, for example a torque or lever force, exerted on the horizontal arm 112 proximate second end 108, which may stop the clamp 102 from returning to the open position. Further, during the transition from the open position to the first closed position, the first protrusion 120 and the second protrusion 122 may converge, which may increase a compressing force, exerted on either side of the surgical tubing 104, which may at least partially restrict a flow of fluid through the surgical tubing 104.

FIG. 3C may show the clamp 102 in a second closed position. For example, in the second closed position, the beveled edge 114 of the horizontal arm 112 may engage a last tooth 126 of the plurality of teeth 118 of the vertical arm 116. As noted above, the beveled edge 114 may act, for example as a pawl, and plurality of the teeth 118 may act, for example, as a rack, as would be understood by a person of ordinary skill in the art.

Thus, in one exemplary embodiment, the clamp 102 may transition from the first closed position as depicted in FIG. 3B to the second closed position as depicted in FIG. 3C by exerting an additional downward force on the horizontal arm 112. For example, an additional downward force may be applied on horizontal arm such that beveled edge 114 may disengage the backside of first tooth 124. Beveled edge 114 may slide down the downward slope of the second tooth to engage the backside of the second tooth. Additional downward pressure may be applied to slide or ratchet beveled edge 114 down each successive tooth of the plurality of teeth 118 until engaging backside of last tooth 126 as would be understood by a person of ordinary skill in the art. In this position, the backside of the last tooth 126 may act against a resilient force, for example a torque or lever force, exerted on horizontal arm 112 proximate second end 108, which may stop clamp 102 from returning to the open position or any other closed position. Further, during the transition from the open position to the first closed position and subsequently to the second closed position, first protrusion 120 and second protrusion 122 may converge with each successive step. Accordingly, an increased compressing force may be exerted on either side of surgical tubing 104, which may at least partially restrict a flow through surgical tubing 104.

Referring generally to FIGS. 3A-C, to transition clamp 102 from a closed position to the open position, a user may exert a force on vertical arm 116 in the direction of first end 106. Beveled edge 114 may disengage from the backside of one of the plurality of teeth 118. A resilient force, for example a torque or lever force, may act on horizontal arm 112 proximate second end 108, to return clamp 102 to the open position.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art. Therefore, the above-described embodiments should be regarded as illustrative rather than restrictive. It should be appreciated that variations to those embodiments can be made by those skilled in the art without departing from the scope of the invention as defined by the following claims.

Having thus described several embodiments for practicing the inventive method, its advantages and objectives can be easily understood. Variations from the description above may and can be made by one skilled in the art without departing from the scope of the invention. Accordingly, this invention is not to be limited by the embodiments as described, which are given by way of example only and not by way of limitation.

What is claimed:

1. A method of and for eliminating noise inherently produced by a vacuum source during a surgical procedure, the method comprising the steps of:
providing a clamp;
connecting the clamp to sterile surgical tubing; and
applying force to the clamp, such that the application of force causes the clamp to transition from an open position to a first closed position, thereby eliminating noise caused by the vacuum created through the sterile surgical tubing during the medical procedure.

2. The method of claim 1, further comprising sterilizing the clamp prior to the initiation of the surgical procedure.

3. The method of claim 1, further comprising maintaining the clamp in a sterile field through the entire surgical procedure.

4. The method of claim 3, wherein the first closed position of the clamp causes the diameter of the sterile surgical tubing to be reduced, but not entirely closed.

5. The method of claim 1, further comprising repositioning the clamp along the length of the sterile surgical tubing.

6. The method of claim 1, wherein repositioning the clamp along the length of the sterile surgical tubing involves sliding the surgical clamp along the length of the sterile surgical tubing.

7. The method of claim 1, wherein the clamp is configured to close, and wherein upon closing of the clamp, the sterile surgical tubing maintains a vacuum within the tubing while reducing or eliminating any noise caused by the vacuum.

8. The method of claim 1, wherein the clamp eliminates suction noise from the surgical procedure.

9. The method of claim 1, wherein the clamp is configured to be opened and closed via a thumb of an operator.

10. The method of claim 1, wherein the surgical clamp comprises:
a distal end,
a hollow body,
a proximal end,
a bottom surface between the distal and the proximal end,
a vertical arm extending from the distal end, the vertical arm having an inner face with a plurality of teeth,
a horizontal arm extending from the proximal end toward the distal end, the horizontal arm having a beveled edge proximal to the inner face,
a first protrusion on the bottom surface extending within the hollow body,
and a second protrusion on the horizontal arm extending within the hollow body;
whereby providing a first force applied to the hollow body such that the horizontal arm and the vertical arm converge to engage the beveled edge with a backside of one of the plurality of teeth, to transition the clamp from an open position to a first closed position of a plurality of plurality positions for restricting or occluding a portion of a medical suction tubing.

11. The method of claim 1, wherein the clamp is serializable.

12. The method of claim 1, wherein the clamp is disposable.

13. The method of claim 1, wherein the clamp is made of materials selected from a group consisting of a polymer or plastic, a metal or metal alloy or a combination thereof.

14. The method of claim 1, wherein the clamp is configured to be captive to the surgical suction tubing.

15. The method of claim 1, wherein the clamp is operable using one hand.

16. A system of and for reducing or eliminating noise inherently produced by a vacuum source during a medical procedure, the system comprising;
a clamp, wherein the clamp includes;
a distal end,
a hollow body,
a proximal end,
a bottom surface between the distal and the proximal end,
a vertical arm extending from the distal end, the vertical arm having an inner face with a plurality of teeth,
a horizontal arm extending from the proximal end toward the distal end, the horizontal arm having a beveled edge proximal to the inner face,
a first protrusion on the bottom surface extending within the hollow body,
and a second protrusion on the horizontal arm extending within the hollow body; whereby a first force applied to the hollow body such that the horizontal arm and the vertical arm converge to engage the beveled edge with a backside of one of the plurality of teeth, to transition the clamp from an open position to a first closed position of a plurality of closed positions for restricting or occluding a portion of a medical suction tubing,
wherein in the closed position, the system reduces or eliminates noise caused by the vacuum source created through the sterile surgical tubing during the medical procedure.

17. The system of claim 16, wherein the clamp is serializable.

18. The system of claim 16, wherein the system eliminates noise from a surgical procedure.
